(19) United States
(12) Patent Application Publication
(10) Pub. No.: US 2025/0213264 A1
(43) Pub. Date: Jul. 3, 2025

(54) HEART VALVE TISSUE STABBING AND CUTTING DEVICE

(71) Applicant: Pi-Cardia Ltd., Rehovot (IL)

(72) Inventors: Benzion Spector, Be’er Yaakov (IL); Or Cohen, Modiin-Macabim-Reut (IL)

(73) Assignee: Pi-Cardia Ltd., Rehovot (IL)

(21) Appl. No.: 19/018,037

(22) Filed: Jan. 13, 2025

Related U.S. Application Data
(60) Provisional application No. 63/616,641, filed on Dec. 31, 2023.

(51) Int. Cl. A61B 17/32; A61B 17/00

(52) U.S. Cl. CPC A61B 17/32; A61B 2017/00477; A61B 2017/320064

(57) ABSTRACT

A heart valve tissue cutting device includes a housing formed with an elongate opening, a cutting guide arm pivoted to the housing by a hinge assembly, and a cutting element including a cutting blade and a stabbing element. The cutting guide arm is formed with a guide slot. The cutting element is arranged to move on a track structure in the housing. A sharp pointed end of the stabbing element is tilted with respect to the cutting blade and points towards the cutting guide arm. The cutting blade is arranged, when in a deployed position, to pass through the guide slot.

### FIELD OF THE INVENTION

The present invention generally relates to devices and methods for transcatheter modification of body tissue such as heart valve leaflets tissue, e.g., aortic, mitral, tricuspid valve leaflets.

### BACKGROUND OF THE INVENTION

PCT Patent Application PCT/IB2020/054729 describes a transcatheter valve laceration device and method. The invention is a method and device, which can be used to perform BASILICA (Bioprosthetic or native Aortic Scallop Intentional Laceration to prevent Iatrogenic Coronary Artery obstruction), or a LAMPOON procedure (for mitral). The device is a cutting or splitting device with attention to preventing damage to neighboring tissues. The device can be implemented in other cardiologic procedures, such as tricuspidization of a bicuspid valve (turning a bicuspid valve into a tricuspid valve by cutting or splitting one of the bicuspid leaflets into two leaflets) or tricuspidization of a quadricuspid valve (lacerating one of the leaflets to turn the valve into a tricuspid valve) or splitting AML (anterior mitral leaflet) to prevent LVOTO (left ventricle outflow tract obstruction), thereby preparing the patient for safe transcatheter aortic or mitral valve replacement (TAVR/TMVR), or for other procedures that involve modifying cardiac/blood vessel tissue.

### SUMMARY

The present invention seeks to provide a device and method for cutting heart valve tissue, such as but not limited to, aortic valve tissue, as is described more in detail hereinbelow. For example, the invention may be used in place of an electrified guidewire (which is the cutter used in prior art BASILICA) to traverse and lacerate the aortic leaflet down the center line.

The device of the invention is particular useful for lacerating the aortic valve leaflets by delivery via the aorta; however, the invention can be used to cut any heart tissue from various approaches and for cutting heart tissue at other valves or portions of the heart.

The term “cutting” refers to any kind of reduction in size or any modification in shape or form, such as but not limited to, cutting, splitting, lacerating, slicing, fracturing, chopping and the like, and the terms are used interchangeably throughout.

There is thus provided in accordance with a non-limiting embodiment of the invention a heart valve tissue cutting device including a housing formed with an elongate opening, a cutting guide arm pivoted to the housing by a hinge assembly, the cutting guide arm being formed with a guide slot, and a cutting element including a cutting blade and a stabbing element, the cutting element being arranged to move on a track structure in the housing, wherein a sharp pointed end of the stabbing element is tilted with respect to the cutting blade and points towards the cutting guide arm, and wherein the cutting blade is arranged, when in a deployed position, to pass through the guide slot. The cutting blade and the stabbing element may be tilted with respect to the cutting guide arm at different angles from each other; e.g., if the cutting blade is oriented as an acute angle, the stabbing element is oriented as an obtuse angle.

In accordance with a non-limiting embodiment of the invention the hinge assembly includes a four-hinge mechanism.

In accordance with a non-limiting embodiment of the invention a blade protector rib is pivoted to the cutting guide arm, and wherein the four-hinge mechanism includes a first hinge link pivoted to a first portion of the housing at a first hinge and pivoted to the blade protector rib at a second hinge, and wherein a second hinge link is pivoted to a second portion of the housing at a third hinge and pivoted to the cutting guide arm at a fourth hinge.

In accordance with a non-limiting embodiment of the invention when the cutting guide arm is pivoted outwards, a portion of the four-hinge mechanism passes through the guide slot.

In accordance with a non-limiting embodiment of the invention a torque biasing device is positioned at a link member of the four-hinge mechanism to deploy the cutting guide arm from a collapsed position to a deployed position.

In accordance with a non-limiting embodiment of the invention the cutting guide arm includes tissue gripping structure for gripping tissue during cutting of the tissue.

### BRIEF DESCRIPTION OF DRAWINGS

The present invention will be understood and appreciated more fully from the following detailed description, taken in conjunction with the drawings in which:

FIG. 1 is a simplified side view illustration of a heart valve tissue cutting device, in accordance with a non-limiting embodiment of the present invention, in a closed (stowed or sheathed) position.

FIGS. 2A and 2B are simplified side view illustrations of the heart valve tissue cutting device, including a cutting guide arm pivoted outwards, in which the cutting guide arm is pivoted more outwards in FIG. 2b than in FIG. 2A.

FIG. 2C is an upper perspective view of the cutting guide arm, showing that it is formed with a slot for guiding the cutting action of a cutting element.

FIG. 3A is a simplified perspective view illustration like FIG. 2B, showing a pivoting cutting element starting to be pivoted and deployed outwards towards the cutting guide arm.

FIG. 3B is an enlarged view of a stabbing element coupled to one end of a cutting blade, in which a sharp pointed end of the stabbing element is tilted with respect to the cutting blade and points towards the cutting guide arm.

FIGS. 3C and 3D are simplified perspective view illustrations of the stabbing element and cutting blade, respectively prior to and after entering a guide slot in the cutting guide arm.

FIG. 3E is an upper perspective view of the stabbing element and cutting blade prior to entering the guide slot in the cutting guide arm.

FIG. 3F is a simplified perspective view illustration of the cutting blade having moved further through the guide slot to cut (e.g., slice) tissue, with the stabbing element and cutting blade being blocked by a blade protector rib which is pivoted to linkage of the cutting guide arm.

FIG. 3G is a simplified perspective view illustration of track structure on which the cutting blade slides.

FIG. 4A is a simplified side view illustration of the cutting blade and stabbing element.

FIGS. 4B and 4C are simplified side view illustrations of a three-hinge, triangular support structure for respectively deploying and collapsing the cutting blade and stabbing element.

FIGS. 4D and 4E are simplified side view illustrations of the three-hinge, triangular support structure, showing two different positions of two lower pins (“lower” in the sense of the drawings) of the triangular support structure sliding in collinear slots of the track structure, while another pin, called a synchronizing pin, slides along a partially open guiding slot, as the triangular support structure lifts the cutting blade.

FIG. 5A is a simplified side view illustration of a torque biasing device (e.g., U-shaped spring) positioned at one of the link members of a four-hinge mechanism to deploy the cutting guide arm from its collapsed position.

FIG. 5B is a simplified side view illustration of the cutting guide arm, showing that it may be constructed of two concentric tubular elements which increases stiffness of the guide arm and provides a hinge support to which the blade protector rib is hinged, and also showing an end cap of the cutting guide arm, and tissue gripping structure of the cutting guide arm, such as a saw tooth edge, for improved gripping of the leaflet tissue during cutting (e.g., splitting) of the tissue.

FIGS. 5C and 5D are simplified illustrations of the cutting guide arm, showing that it may be constructed of first and second concentric semi-tubular elements.

FIGS. 6A and 6B are simplified pictorial illustrations of the heart valve tissue cutting device, delivered to the heart and positioned to cut heart valve tissue.

### DETAILED DESCRIPTION

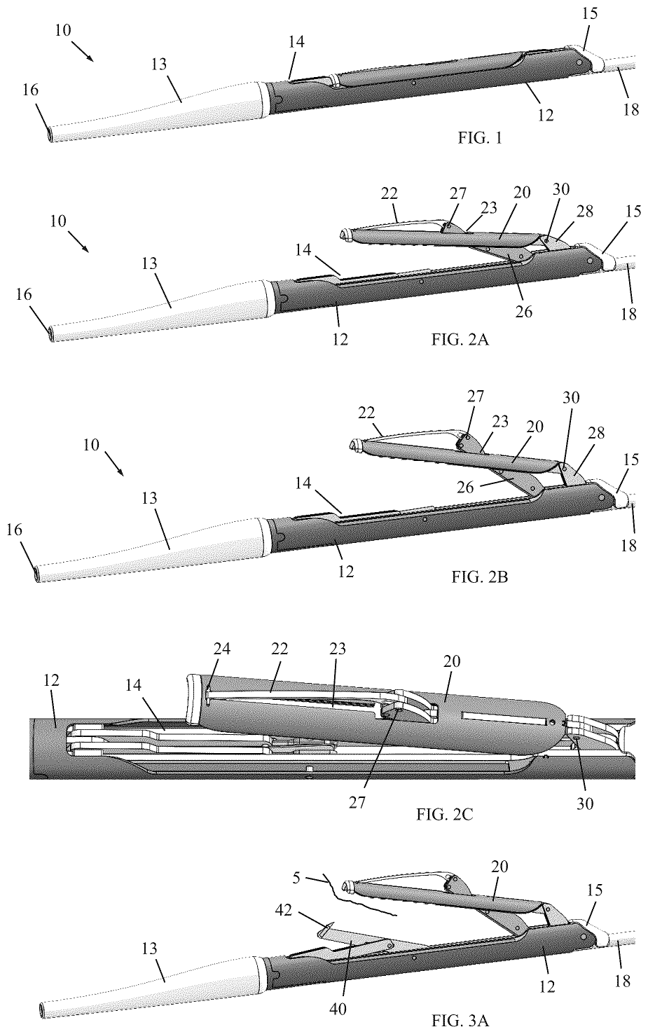

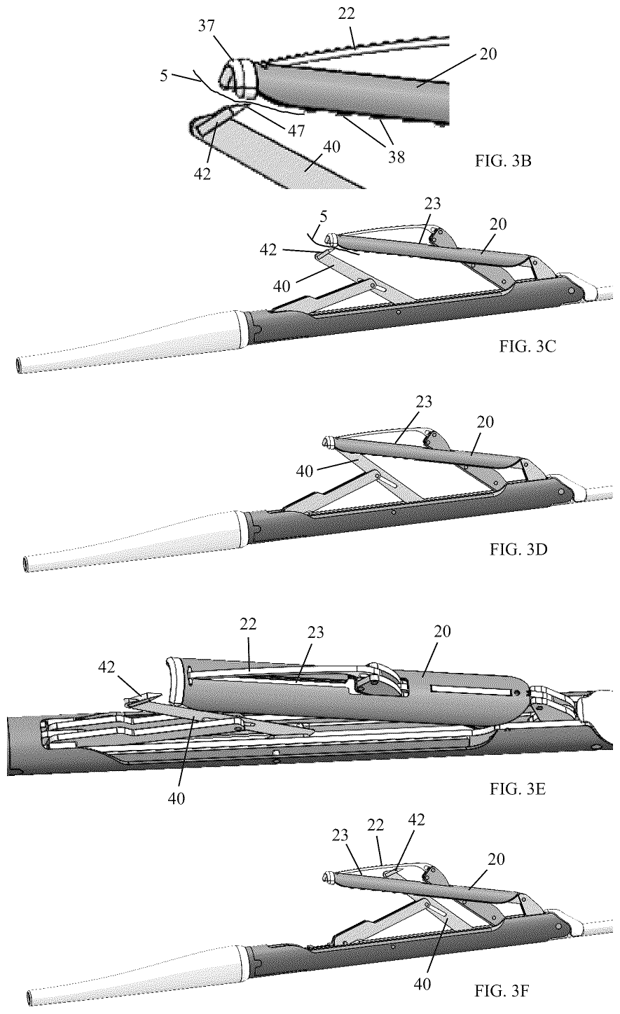

Reference is now made to FIGS. 1 and 2A-2C, which illustrate a heart valve tissue cutting device 10, in accordance with a non-limiting embodiment of the present invention.

The heart valve tissue cutting device 10 is particularly useful for cutting aortic valve tissue (such as aortic valve leaflets) and can be delivered via the aorta to cut the aortic valve tissue by initially stabbing or puncturing the tissue with a stabbing element of a cutting blade, as described below, and then axially slicing the tissue by moving the cutting blade, such as in the direction back to the aorta. For such an approach, the distal end is the left end in the drawings and the proximal end is the right end in the drawings. It should be understood that distal and proximal are relative, non-limiting terms, and the invention can be used in other orientations. The invention is not limited to the aortic valve leaflets. The device of the invention can be implemented in other cardiologic procedures, such as cutting or slicing mitral valve leaflets, tricuspidization of a bicuspid valve (turning a bicuspid valve into a tricuspid valve by cutting or splitting one of the bicuspid leaflets into two leaflets) or tricuspidization of a quadricuspid valve (lacerating one of the leaflets to turn the valve into a tricuspid valve) or splitting AML (anterior mitral leaflet) to prevent LVOTO (left ventricle outflow tract obstruction), thereby preparing the patient for safe transcatheter aortic or mitral valve replacement (TAVR/TMVR), or for other procedures that involve modifying cardiac/blood vessel tissue.

The heart valve tissue cutting device 10 may include a (e.g., slender, cylindrical) housing 12 formed with an elongate opening 14, which may have rounded ends. The housing 12 may be made of medical grade stainless steel or any other suitable material.

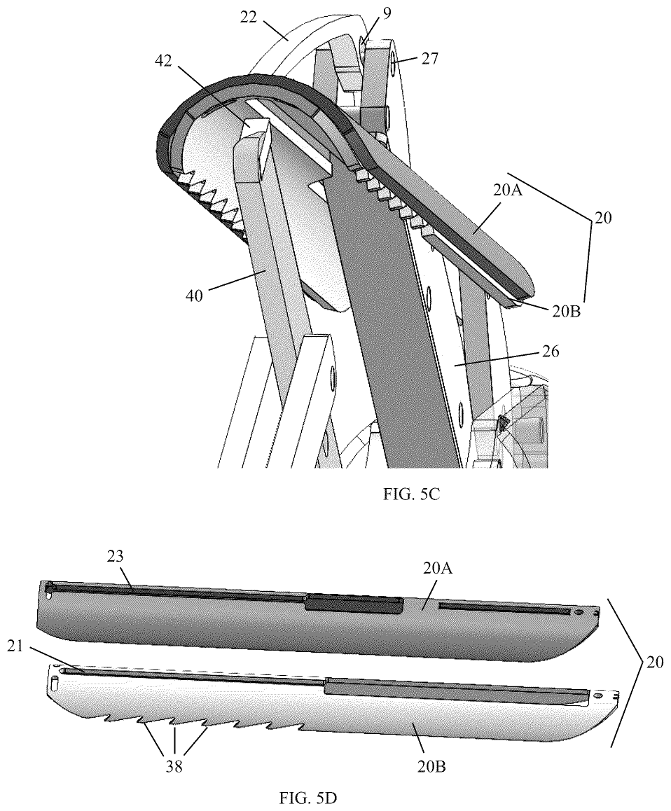

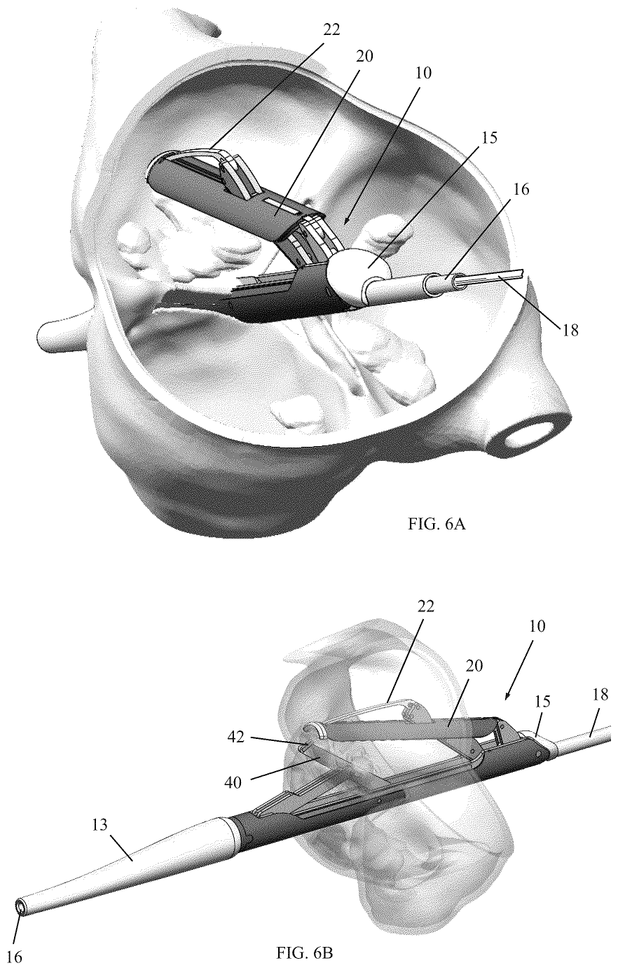

Housing 12 may include a guidewire lumen 16 along a lower axial length of housing 12 (“lower” in the sense of the drawings; not necessarily lower when the device is being used), as seen best in FIG. 5B. The proximal end of guidewire lumen 16 is seen at the right end in the non-limiting sense of the drawing in FIG. 6A. Accordingly, device 10 can be delivered over a guidewire that passes though guidewire lumen 16. (The guidewire, not shown, may pass through or next to actuator wire or push rod 18, described below.)

The housing 12 may include first and second end caps 13 and 15, as seen in FIGS. 1, 2A and 2B. The first end cap 13 (which is distal in the non-limiting sense of the drawings) may have a tapered shape such that the first end cap 13 is narrow at the distal end. This may help in inserting the device through tissue or body lumens. The second end cap 15 may have a tilted portion that is tilted towards the direction of first end cap 13.

The heart valve tissue cutting device 10 may include a cutting guide arm 20 pivoted to housing 12 by a hinge assembly (as described below with reference to FIGS. 5A and 5B). A blade protector rib 22 may be pivoted to cutting guide arm 20 at a pivot or hinge 24 (FIG. 2C). A guide slot 23 may be formed in cutting guide arm 20 for guiding the cutting action of a cutting element, described hereinbelow. As seen in FIG. 2C, blade protector rib 22 may be configured to enter and exit the guide slot 23.

Reference is now made to FIGS. 5A and 5B. The cutting guide arm 20 may be pivoted to housing 12 by means of a four-hinge mechanism. A first hinge link 26 may be pivoted to a first portion of housing 12 at a first hinge (pivot) 25, and pivoted to blade protector rib 22 at a second hinge (pivot) 27. Second hinge 27 may be a sliding hinge, in which a pin slides in a channel 9 formed in blade protector rib 22. A second hinge link 28 may be pivoted to a second portion of housing 12 (proximal to the first portion in the non-limiting sense of the drawings), at a third hinge (pivot) 29, which is proximal to first hinge 25, and pivoted to cutting guide arm 20 at a fourth hinge (pivot) 30. As seen in FIG. 5A, a third hinge link 31 may be pivoted to second hinge link 28 at fourth hinge 30 and to an intermediate portion of first hinge link 26 at a fifth hinge (pivot) 32.

As seen in FIGS. 5A and 5B, a torque biasing device 33 (e.g., U-shaped spring) may be positioned at one of the link members of the four-hinge mechanism, such as second hinge link 28, to deploy the cutting guide arm 20 from its collapsed position. One end of torque biasing device 33 may be coupled to pivot 29 and another end may be coupled to a pivot 34. An intermediate portion of torque biasing device 33 may be constrained to move in a slot 35 by means of a pin 36.

The cutting guide arm 20 may be initially in a collapsed position when the device is first introduced into the body, such as in a delivery sheath (not shown). When the delivery sheath is removed, torque biasing device 33 applies a spring force to outwardly deploy the cutting guide arm 20 from its collapsed position, activating the four-hinge mechanism.

Reference is made particularly to FIGS. 5C and 5D. Cutting guide arm 20 may be constructed of first and second concentric semi-tubular elements 20A and 20B. In the non-limiting sense of the drawing, they are the upper and lower semi-tubular elements 20A and 20B. In this construction, guide slot 23 is formed in first (upper) semi-tubular element 20A, and another slot 21 is formed in second (lower) semi-tubular element 20B, so that first link member 26 (FIG. 5C) passes through guide slot 23 and slot 21. This construction increases stiffness of the cutting guide arm 20 and provides the hinge 24 to which the blade protector rib 22 is hinged (22 and 24 shown in FIG. 5B). The reverse U-shape of the cutting guide arm 20 is designed to guide the blade tip into guide slot 23 if the lift action is not centered due to tolerances.

A semi-circular end cap 37 may be coupled to the end (in the non-limiting sense of the drawings, the distal end) of cutting guide arm 20. End cap 37 may be constructed as an over-mold cushion, made from a polymeric or elastomeric material.

Cutting guide arm 20 may include tissue gripping structure 38, such as a saw tooth edge, for improved gripping of the leaflet tissue during cutting (e.g., splitting) of the tissue. The tissue gripping structure 38 may be formed along the lower edge of either one or both of first and second concentric semi-tubular elements 20A and 20B.

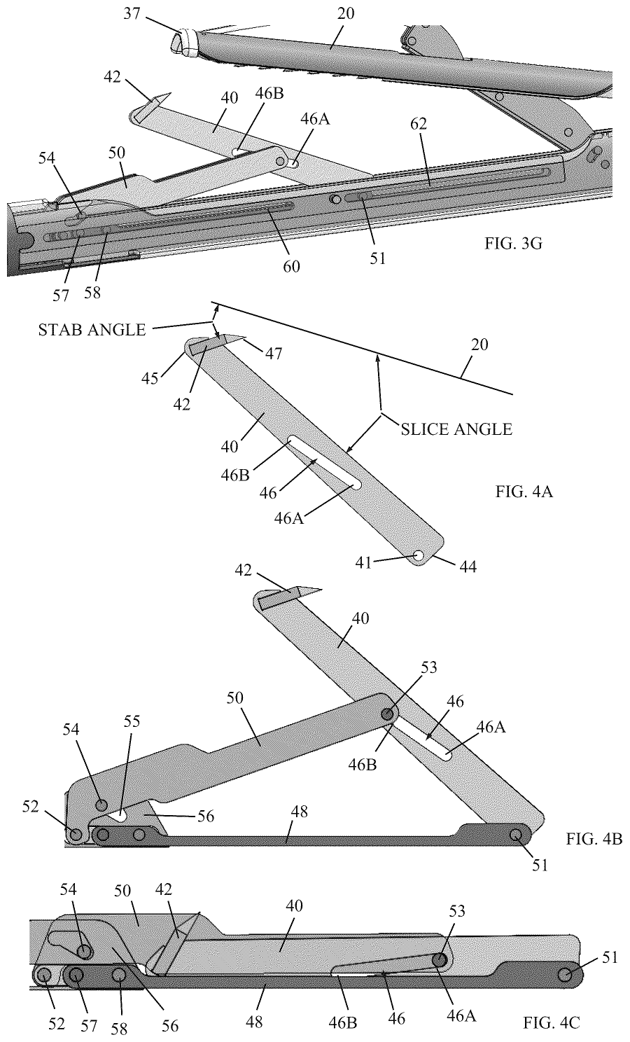

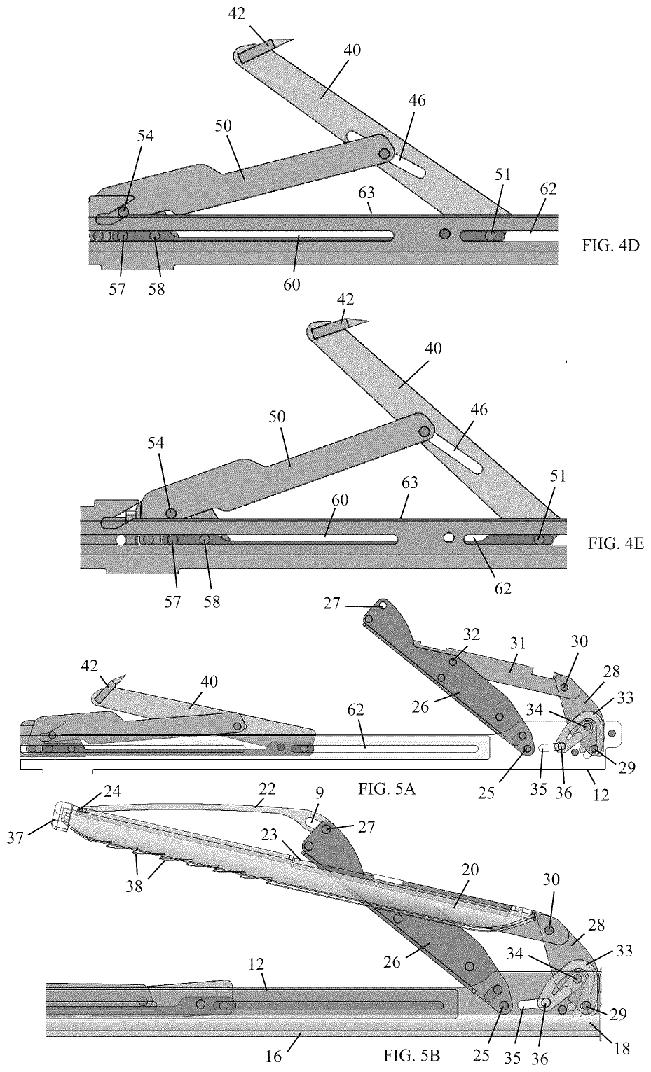

Reference is now made to FIG. 4A, which illustrates a cutting blade 40 and stabbing element 42 of the device. Cutting blade 40 may include a cutting edge 43 that extends from a first end 44 (e.g., the proximal end) to a second end 45 (e.g., the distal end). Cutting blade 40 may be formed with a slot 46 which has a first end 46A (e.g., the proximal end) and a second end 46B (e.g., the distal end). Slot 46 is tilted such that first end 46A is closer to cutting guide arm 20 than second end 46B, as seen in FIG. 3G. In the non-limiting sense of the drawings, when cutting blade 40 is in the collapsed position, first end 46A is higher than second end 46B. This results from the triangular, 3-axis support structure.

The stabbing element 42 is coupled to the second end 45 of cutting blade 40. A sharp pointed end 47 of stabbing element 42 is tilted with respect to cutting blade 40 and points towards the cutting guide arm 20, as seen in FIG. 3B. As seen in FIG. 4A, the stabbing angle (the angle between the stabbing element 42 and cutting guide arm 20) is oriented in a direction opposite to the slicing angle (the angle between the cutting blade 40 and cutting guide arm 20); e.g., if the cutting blade is oriented as an acute angle, the stabbing element is oriented as an obtuse angle. The leaflet is sandwiched between the cutting element and the cutting guide arm 20, with the cutting guide arm 20 acting as an anvil. The angle for stabbing or puncturing the tissue is different than the slicing angle so that the leaflet tissue is initially acquired and punctured/stabbed by stabbing element 42, which cannot successfully be done by the cutting blade 40. Afterwards, the cutting blade 40 is at the correct angle for slicing the tissue as the blade moves forward.

Reference is now made to FIGS. 4B and 4C, which illustrate a three-hinge, triangular support structure for respectively deploying and collapsing cutting blade 40 and stabbing element 42.

The three-hinge, triangular support structure may include the cutting blade 40, a base member 48 and a cutting blade link member 50. Cutting blade 40 may be pivoted to base member 48 at a first pivot (pin) 51. (Pin 51 may be coupled to a hole 41 of cutting blade, seen in FIG. 4A.) Cutting blade link member 50 may be pivoted to base member 48 at a second pivot 52. Cutting blade link member 50 may be pivoted to slot 46 of cutting blade 40 by a pin 53. Cutting blade link member 50 may be constrained in its angular pivoting movement by a pin 54 that slides in a track slot 55 of a lug 56 which is coupled to base member 48. In the deployed position of FIG. 4B, pin 53 of cutting blade link member 50 is at second end 46B of slot 46; in the collapsed position of FIG. 4C, pin 53 of cutting blade link member 50 is at first end 46A of slot 46. Lug 56 may be coupled to base member 48 by means of first and second coupling pins 57 and 58.

Reference is now made to FIGS. 4D and 4E, which illustrate two different positions of the cutting blade 40 in its axial travel. The first and second coupling pins 57 and 58 are arranged to travel in a first track slot 60, and the first pivot (pin) 51 is arranged to travel in a second track slot 62. The first and second track slots 60 and 62 may be collinear, but not necessarily. Pin 54, which may be called a synchronizing pin, slides along a partially open guiding slot 63, as the triangular support structure lifts the cutting blade 40.

Cutting blade 40 may be moved in its axial travel by an actuator wire or push rod 18 (FIGS. 1, 2A, 2B, and 6A)) that passes through or near base element 48 (e.g., to pin 52 or pin 51 or to any other suitable portion of base element 48, lug 56 or other portion of the triangular support structure). The actuator wire 18 may be coupled to a manipulating device (not shown) used by the surgeon.

Reference is now made to FIGS. 3A-3G, which illustrate use of the device.

In FIG. 3A, the pivoting cutting element (cutting blade 40 and stabbing element

42) starts to pivot and deploy outwards towards cutting guide arm 20. Since the cutting guide arm 20 is spring-loaded as described above, it deploys outwards upon unsheathing. The cutting guide arm 20 is positioned over the leaflet at the base location where the operator wishes to start the cutting operation.

FIG. 3B shows the sharp pointed end 47 of stabbing element 42 tilted with respect to cutting blade 40 so that it points towards the cutting guide arm 20. The tilted orientation of stabbing element 42 enables it to acquire a leaflet tissue 5 and puncture it, so that afterwards the cutting blade 40 can penetrate and cut (slice) the leaflet tissue 5. If the cutting blade 40 were used to stab or puncture the leaflet without the stabbing element 42, the leaflet could slip over the sharp blade tip and may not get punctured.

FIGS. 3C and 3D illustrate stabbing element 42 and cutting blade 40, respectively prior to and after entering guide slot 23 in the cutting guide arm 20. The cutting guide arm 20 acts as a cutting anvil. The angle of tilt A (FIG. 3D, called the slice angle in FIG. 4A) between the cutting blade 40 and the cutting guide arm 20 is designed to achieve a smooth slicing action while the cutting blade 40 slides (e.g., proximally) and pushes the leaflet towards the cutting guide arm 20.

The engagement of the cutting blade 40 into the slot 23 of cutting guide arm 20 facilitates the slicing action of the leaflet that is performed against the cutting guide arm 20. This engagement keeps the leaflet engaged with the device until the cutting (slicing) action is completed (e.g., a complete split of the leaflet). The device does not lose its hold of the leaflet during the cutting action.

FIG. 3E illustrates stabbing element 42 and cutting blade 40 prior to entering guide slot 23 in cutting guide arm 20.

FIG. 3F illustrates cutting blade 40 having moved further through guide slot 23 to cut (e.g., slice) tissue, with the stabbing element 42 and cutting blade 40 being blocked by blade protector rib 22 which is pivoted to linkage of cutting guide arm 20. This prevents injuring tissue near the movement area of stabbing element 42.

FIG. 3G illustrates the track structure on which cutting blade 40 slides, including first and second coupling pins 57 and 58 that travel in first track slot 60, first pivot (pin) 51 that travels in second track slot 62, and synchronizing pin 54 that slides along guiding slot 63 (FIG. 4D), as the triangular support structure lifts cutting blade 40.

Reference is now made to FIGS. 6A and 6B, which illustrate the heart valve tissue cutting device 10 delivered to the heart and positioned to cut heart valve tissue.

It is noted that the invention can also be carried out with an electric cutting element (e.g., electrified blade element or electric lacerating wire).

1. A heart valve tissue cutting device comprising:
a housing formed with an elongate opening;
a cutting guide arm pivoted to said housing by a hinge assembly, said cutting guide arm being formed with a guide slot; and
a cutting element comprising a cutting blade and a stabbing element, said cutting element being arranged to move on a track structure in said housing, wherein a sharp pointed end of said stabbing element is tilted with respect to said cutting blade and points towards said cutting guide arm, and wherein said cutting blade is arranged, when in a deployed position, to pass through said guide slot.

2. The heart valve tissue cutting device according to claim 1, wherein said cutting blade and said stabbing element are tilted with respect to said cutting guide arm at different angles from each other.

3. The heart valve tissue cutting device according to claim 1, wherein said hinge assembly comprises a four-hinge mechanism.

4. The heart valve tissue cutting device according to claim 1, wherein a blade protector rib is pivoted to said cutting guide arm.

5. The heart valve tissue cutting device according to claim 1, wherein when said cutting guide arm is pivoted outwards, a portion of said four-hinge mechanism passes through said guide slot.

6. The heart valve tissue cutting device according to claim 1, wherein said cutting guide arm is constructed of first and second concentric semi-tubular elements.

7. The heart valve tissue cutting device according to claim 3, wherein a torque biasing device is positioned at a link member of said four-hinge mechanism to deploy said cutting guide arm from a collapsed position to a deployed position.

8. The heart valve tissue cutting device according to claim 1, wherein said cutting guide arm comprises tissue gripping structure for gripping tissue during cutting of the tissue.

9. The heart valve tissue cutting device according to claim 8, wherein said tissue gripping structure comprises a saw tooth edge.

10. The heart valve tissue cutting device according to claim 1, wherein said cutting guide arm comprises a semi-tubular, U-shaped structure.

11. The heart valve tissue cutting device according to claim 1, wherein said cutting element comprises an electric cutting element.

12. A method for cutting heart valve tissue comprising using the device of claim 1 to grasp a leaflet between said cutting guide arm and said housing, and while the leaflet is grasped, moving the cutting element to cut the leaflet.

13. The method according to claim 12, wherein said stabbing element punctures the leaflet and forms a starting point for said cutting blade to pass through and cut the leaflet.

14. The method according to claim 12, wherein said stabbing element prevents the leaflet from sliding off the cutting blade.

15. The method according to claim 12, wherein the leaflet is sandwiched between said cutting element and said cutting guide arm, with said cutting guide arm acting as an anvil.
